(19) United States
(12) Patent Application Publication
(10) Pub. No.: US 2013/0263856 A1
(43) Pub. Date: Oct. 10, 2013

(54) ENTRAINMENT COVER AND INTAKE MODULE HAVING THE SAME

(71) Applicant: GALEMED CORPORATION, I-Lan Hsien (TW)

(72) Inventors: Gary C.J. Lee, I-Lan Hsien (TW); Ding-Yang Hsu, Taipei City (TW)

(73) Assignee: GALEMED CORPORATION, I-Lan Hsien (TW)

(21) Appl. No.: 13/735,202

(22) Filed: Jan. 7, 2013

(30) Foreign Application Priority Data
Apr. 9, 2012 (TW) 101112448

(51) Int. Cl. A61M 16/00

(52) U.S. Cl. CPC A61M 16/0096
USPC 128/204.25

(57) ABSTRACT

An intake module includes an outer tube body defining an intake chamber and having an intake end, and an inner tube body disposed in the outer tube body and having an intake hole communicating with the intake chamber. An intake lid covers the intake end and has an inlet hole for allowing the intake chamber to be in fluid communication with the outside so that a compressed gas is fed into the inlet hole. A needle tube defines a jet hole for spraying the compressed gas from the needle tube into the intake hole. When the compressed gas is delivered from the intake lid into the inner tube body, air is drawn into the intake hole to mix with the compressed gas according to Venturi effect.

### CROSS-REFERENCE TO RELATED APPLICATION

This application claims priority of Taiwanese Application No. 101112448, filed on Apr. 9, 2012.

### BACKGROUND OF THE INVENTION

1. Field of the Invention

The invention relates to an intake module, more particularly to an intake module having an entrainment cover by which ambient air is entrained to increase the total flow rate of the intake module.

2. Description of the Related Art

A current respiratory device typically includes a respiratory mask coupled to a compressed oxygen cylinder by an intake module for entraining ambient air into the compressed oxygen to dilute the oxygen concentration and increase the total flow rate of the gas flowing into the respiratory mask. However, the diversion mechanisms for entraining ambient air in current intake modules are unable to steadily entrain ambient air into the compressed oxygen stream, making the current mechanisms inefficient for increasing the total flow rate.

### SUMMARY OF THE INVENTION

Therefore, an object of the present invention is to provide an intake module for steadily and uniformly entraining ambient air into the compressed oxygen stream to increase the total flow rate of the gas flowing into the respiratory mask.

According to an aspect of the present invention, there is provided an intake module comprising an air delivery tube, an intake lid, an entrainment cover, and a needle tube.

The air delivery tube includes an outer tube body and an inner tube body disposed in the outer tube body. The outer tube body includes an intake end, an exhaust end opposite to the intake end, an intake chamber defined by the outer tube body, and an intake port that is formed between the intake end and the exhaust end and that exposes the intake chamber. The inner tube body extends from the exhaust end towards the intake end, and includes an exhaust hole disposed at an end thereof and adjoining the exhaust end, and an intake hole disposed at an opposite end thereof and in fluid communication with the intake chamber.

The intake lid covers the intake end, seals the intake chamber, and includes an inlet hole that extends through the intake lid and allows the intake chamber to be in fluid communication with the atmosphere. The entrainment cover is sleeved on the end of the inner tube body formed with the intake hole, and has a positioning hole formed therethrough and located between the intake hole and the inlet hole, and an entrainment port for establishing fluid communication between the intake chamber and the intake hole of the inner tube body. The needle tube establishes fluid communication between the inlet hole of the intake lid and the positioning hole of the entrainment cover, and defines a jet hole for spraying a compressed gas from the needle tube into the intake hole. The diameter of the jet hole is less than that of the intake hole of the inner tube body.

Preferably, the entrainment cover is a hollow and generally conical, and includes a securing ring portion that is sleeved fixedly on the inner tube body, a tapered portion that extends and tapers from the securing ring portion towards the intake end, and an end wall at a tip of the tapered portion. The entrainment port is formed in the tapered portion. The positioning hole is formed in the end wall. The entrainment cover defines a mixing space that is in fluid communication with the intake hole, the entrainment port, and the jet hole.

Additionally, the air delivery tube defines a central axis (L), about which the inner tube body and the jet hole are centered.

Preferably, the intake lid further includes a lid body sealably engaging the intake end of the outer tube body, and a rod extending from the lid body towards the inner tube body. The inlet hole extends through the rod and the lid body, is centered about the central axis (L), and has a first hole portion proximal to the lid body for is adapted to be in fluid communication with a compressed gas source, and a second hole portion distal from the lid body and having a diameter smaller than that of the first hole portion. The needle tube extends through the second hole portion.

According to another aspect of the present invention, there is provided an entrainment cover by which ambient air can be steadily and uniformly entrained into the compressed gas stream to increase the total flow rate of the gas flowing into the respiratory mask.

The entrainment cover is a hollow and conical, is sleeved fixedly on the inner tube body, and has a positioning hole for establishing fluid communication between the intake hole and the inlet hole via the needle tube. The entrainment cover further has an entrainment port for establishing fluid communication between the intake chamber and the intake hole of the inner tube body, and defines a mixing space that is in fluid communication with the inlet hole, the entrainment port, and the jet hole.

Preferably, the entrainment cover further includes a securing ring portion sleeved fixedly on the inner tube body, a tapered portion extending and tapering from the securing ring portion towards the intake end, and an end wall at a tip of the tapered portion. The entrainment port is formed in the tapered portion. The positioning hole is formed in the end wall.

The efficacy of this invention resides in the needle tube and the entrainment cover. Specifically, the needle tube is fixed between the positioning hole of the entrainment cover and the inlet hole, so as to provide a steady stream of compressed gas into the intake module along the central axis. The entrainment cover is sleeved fixedly the inner tube body, and allows for steady and uniform entrainment of ambient air into the compressed gas stream provided by the needle tube to achieve an increased total flow rate of the gas flowing into the respiratory mask.

### BRIEF DESCRIPTION OF THE DRAWINGS

Other features and advantages of the present invention will become apparent in the following detailed description of a preferred embodiment with reference to the accompanying drawings, of which:

FIG. 1 is an exploded perspective view of the preferred embodiment of an intake module of the present invention;

FIG. 2 is an assembled perspective view of the preferred embodiment; and

FIG. 3 is a sectional view of the preferred embodiment.

### DETAILED DESCRIPTION OF THE PREFERRED EMBODIMENT

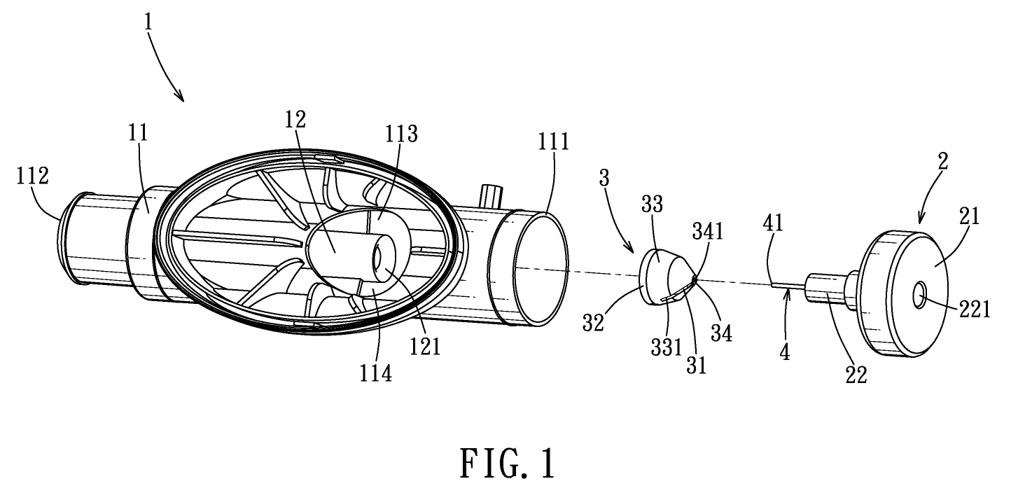

With reference to FIG. 1, the preferred embodiment of an intake module of the present invention is coupled to a compressed oxygen cylinder (not shown) via a conduit tube (not shown), and is connected with a respiratory mask (not shown) via a flexible pipe (not shown). The intake module receives compressed oxygen from the compressed oxygen cylinder, and passes it through the flexible pipe and into the respiratory mask for inspiration by a user.

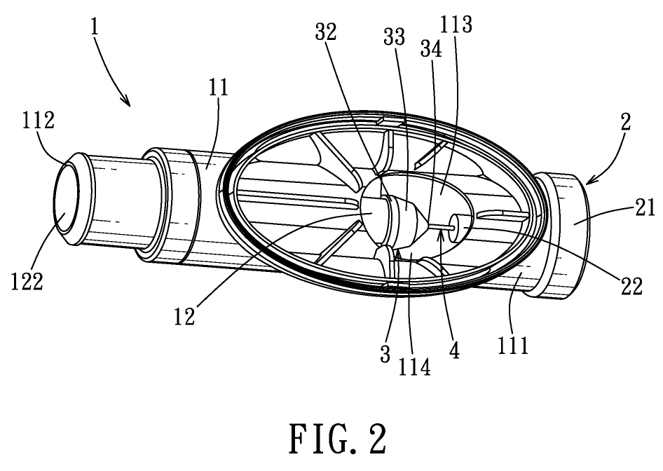

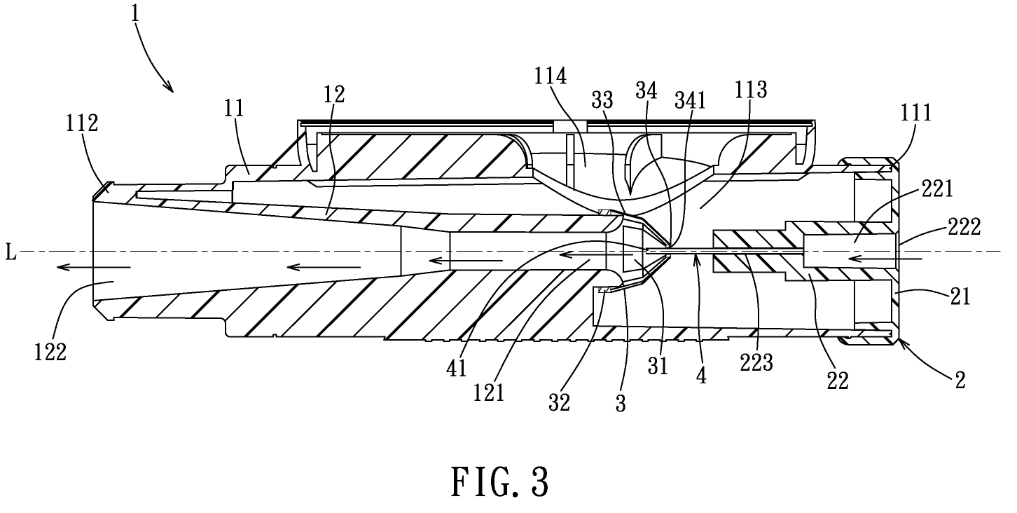

With additional reference to FIGS. 2 and 3, the intake module comprises an air delivery tube 1 having a central axis (L), an intake lid 2 sealably engaging an end of the air delivery tube 1, a hollow, generally conical entrainment cover 3 disposed within the air delivery tube 1, and a needle tube 4 for establishing fluid communication between the intake lid 2 and the air delivery tube 1.

The air delivery tube 1 includes an outer tube body 11 and an inner tube body 12 disposed in the outer tube body 11, both of which are centered about the central axis (L). The outer tube body 11 has an intake end 111 that is engageable with the intake lid 2, an exhaust end 112 opposite to the intake end 111, an intake chamber 113 defined by the outer tube body 11 and open at the intake end 111, and an intake port 114 that is formed in the outer tube body 11 between the intake end 111 and the exhaust end 112 and that exposes the intake chamber 113 to the atmosphere. The compressed oxygen flows into the air delivery tube 1 through the intake end 111 of the outer tube body 11. Ambient air flows into the intake chamber 113 through the intake port 114. The inner tube body 12 extends from the exhaust end 112 towards the intake end 111, and includes an exhaust hole 122 that is disposed at an end thereof and that is defined by the exhaust end 112, and an intake hole 121 that is disposed at an opposite end thereof and that is in fluid communication with the intake chamber 113.

The intake lid 2 includes a lid body 21 for sealably engaging the intake end 111 of the outer tube body 11, a rod 22 extending from the lid body 21 towards the inner tube body 12, and an inlet hole 221 extending through the rod 22 and the lid body 21 along the central axis (L). The inlet hole 221 has a first hole portion 222 proximate to the lid body 21, and a second hole portion 223 distal from the lid body 21. The diameter of the second hole portion 223 is less than that of the first hole portion 222.

The entrainment cover 3 includes a securing ring portion 32 securely sleeved on the end of the inner tube body 12 formed with the intake hole 121, a tapered portion 33 extending and tapering from the securing ring portion 32 towards the intake end 111, and an end wall 34 at a tip of the tapered portion 33. A positioning hole 341 is formed in the end wall 34 for receiving one end of the needle tube 4. An entrainment port 331 is formed in the tapered portion 33 and configured as a rectangular-cross-sectioned slot extending from the middle of the tapered portion 33 to the end wall 34 for establishing fluid communication between the intake chamber 113 and the intake hole 121 of the inner tube body 12. The size of the entrainment port 331 can be varied during manufacture of the entrainment cover 3 to adjust the amount of ambient air to entrain. In addition, the shape and number of the entrainment port 331 are not limited to those in the preferred embodiment and may be modified as necessary to achieve desired results.

Further, the entrainment cover 3 defines a mixing space 31 that is in fluid communication with a jet hole 41 of the needle 4, the entrainment port 331, and the intake hole 121 and that receives the compressed oxygen flowing from the jet hole 41 and ambient air flowing from the entrainment port 331 to decrease the concentration of oxygen, so as to direct the mixture of the compressed oxygen with ambient air into the intake hole 121.

The jet hole 41 has a diameter less than those of the conduit tube and the intake hole 121 of the inner tube body 12 for spraying the compressed oxygen from the needle tube 4 into the mixing space 31. In this embodiment, one end of the needle tube 4 is inserted through the second hole portion 223 of the inlet hole 221, while the other end is inserted through the positioning hole 341 and into the fixedly extends along the central axis (L).

Operation of the intake module of the present invention requires the conduit tube to be disposed between and in fluid communication with the compressed oxygen cylinder and the first hole portion 222 of the inlet hole 221 so that the compressed oxygen flows through the inlet hole 221 out of the jet hole 41 of the needle tube 4. Because the diameter of the conduit tube is greater than that of the jet hole 41, the velocity of the compressed oxygen increases and the pressure of the compressed oxygen decreases as it flows through the jet hole 41. The jet hole 41 directs the flow of compressed oxygen into the mixing space 31 of the entrainment cover 3 towards the intake hole 121 of the inner tube body 12. Specifically, when the mixing space 31 receives the stream of compressed oxygen from the jet hole 41, the compressed oxygen stream flows at high velocity and low pressure compared to the ambient air within the mixing space 31. Via the Venturi effect, the pressure difference causes the stream of compressed oxygen to entrain ambient air in the mixing space 31 as it flows into the intake hole 121.

The entrainment of ambient air into the compressed oxygen results in a decrease in the oxygen concentration of the gas flowing through the mixing space 31, and an increase in the t the gas within the stream. This mixture flows through the inner tube body 12 and out of the intake module from the exhaust hole 122 into the flexible pipe.

Importantly, the entrainment cover 3 firmly secures the inner tube body 12 and the needle tube 4 along the central axis (L) for optimally introducing ambient air into the intake hole 121 of the inner tube body 12 via the Venturi effect. The compressed oxygen stream steadily and uniformly entrains ambient air, incorporating it therein while flowing to the intake hole 121, thus increasing the total flow rate of the gas flowing from the intake module into the respiratory mask.

Further, due to the Venturi effect, when ambient air from the mixing space 31 is entrained by the compressed oxygen into the intake hole 121, the air pressure within the mixing space 31 decreases. The resulting pressure difference causes the air outside of the outer tube body 11 to enter the intake chamber 113 through the intake port 114. This ambient air flows from the intake port 114 into the mixing space 31 through the entrainment port 331 to replenish the mixing space 31 with air.

To sum up, the fixed position of the needle tube 4 between the inlet hole 221 of the intake lid 2 and the positioning hole 341 of the entrainment cover 3 along the central axis (L) allows the compressed oxygen to flow into the mixing space 31 and, via the Venturi effect, steadily and uniformly entrain ambient air as it flows through the mixing space 31. The addition of ambient air into the compressed oxygen stream increases the total flow rate and thus fulfills the purpose of this invention.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. An intake module comprising:
an air delivery tube including an outer tube body and an inner tube body disposed in said outer tube body, said outer tube body having an intake end, an exhaust end opposite to said intake end, an intake chamber that is defined by said outer tube body and that is open at said intake end, and an intake port that is formed between said intake end and said exhaust end and that exposes said intake chamber, said inner tube body extending from said exhaust end towards said intake end and having an exhaust hole disposed at an end thereof and adjoining said exhaust end, and an intake hole disposed at an opposite end thereof and in fluid communication with said intake chamber;
an intake lid covering said intake end and sealing said intake chamber, said intake lid having an inlet hole adapted for allowing said intake chamber to be in fluid communication with the outside therethrough so that a compressed gas is fed into said inlet hole;
an entrainment cover sleeved on the end of said inner tube body formed with said intake hole and having a positioning hole formed therethrough and disposed between said inlet hole and said intake hole, and an entrainment port disposed between and in fluid communication with said intake chamber and said intake hole; and
a needle tube extending into said inlet hole and said positioning hole and defining a jet hole adapted for spraying the compressed gas from said needle tube into said intake hole in said inner tube body of said air delivery tube, said jet hole having a diameter less than that of said intake hole of said inner tube body;
wherein, when the compressed gas is delivered from said intake lid into said inner tube body of said air delivery tube, air is drawn into said intake hole to mix with the compressed gas according to Venturi effect.

2. The intake module in claim 1, wherein said entrainment cover is hollow and generally conical, and defines a mixing space in fluid communication with said intake hole, said entrainment port, and said jet hole.

3. The intake module in claim 2, wherein said entrainment cover has a securing ring portion sleeved fixedly on said inner tube body, a tapered portion extending and tapering from said securing ring portion toward the intake end, and an end wall disposed at a tip of said tapered portion, said entrainment port being formed in said tapered portion, said positioning hole being formed of said end wall.

4. The intake module in any of claim 1, wherein said air delivery tube defines a central axis, said inner tube body and said jet hole being centered about said central axis.

5. The intake module in claim 4, wherein said intake lid includes a lid body engaging said outer tube body and a rod extending from said lid body toward said intake hole of said inner tube body, said inlet hole extending along said central axis through said rod and said lid body, said inlet hole having a first hole portion proximal to said lid body along said central axis and a second hole portion distal from said lid body along said central axis, said second hole portion having a diameter smaller than that of said first hole portion, said needle tube extending through said second hole portion.

6. An entrainment cover adapted for use in an intake module, the intake module including an air delivery tube having an outer tube body, an inner tube body disposed the outer tube body, and a needle tube disposed within the outer tube body and having a jet hole, said outer tube body defining an intake chamber, said inner tube body having an intake hole in fluid communication with the intake chamber, said entrainment cover being hollow and generally conical and being adapted to be disposed between the inner tube body and the needle tube, said entrainment cover being sleeved fixedly on the inner tube body and comprising a positioning hole adapted to permit the needle tube to be inserted therethrough, an entrainment port adapted to be disposed between and in fluid communication with the intake chamber and the intake hole, and a mixing space adapted to be in fluid communication with the intake hole, said entrainment port, and the jet hole.

7. The entrainment cover of claim 6, further comprising a securing ring portion adapted to be sleeved fixedly on the inner tube body, a tapered portion extending and tapering from said securing ring portion toward the intake end, and an end wall disposed at a tip of said tapered portion, said entrainment port formed in said tapered portion, said positioning hole being formed in said end wall.
